(19) United States
(12) Patent Application Publication
(10) Pub. No.: US 2021/0361628 A1
(43) Pub. Date: Nov. 25, 2021

(54) 2-OXOTHIAZOLE COMPOSITIONS FOR TREATMENT OF FIBROTIC DISEASE

(71) Applicant: Avexxin AS, Avexxin AS (NO)

(72) Inventors: Astrid J. Feuerherm, Trondheim (NO); Berit Johansen, Trondheim (NO)

(21) Appl. No.: 17/050,071

(22) PCT Filed: Apr. 24, 2019
(86) PCT No.: PCT/EP2019/060544

§ 371 Date: Oct. 23, 2020

(30) Foreign Application Priority Data
Apr. 24, 2018 (GB) 1806663.9

(51) Int. Cl. A61K 31/426; A61P 19/04

(52) U.S. Cl. CPC A61K 31/426; A61P 19/04

(57) ABSTRACT

A compound of formula (I), (I) wherein R¹⁰ is H or C₁₋₆ alkyl such as Me; R₂ is H, —(CH₂)ₚCOOH, —(CH₂)ₚCON(R⁵)₂ or —(CH₂)ₚCOOC₁₋₆alkyl; each R⁵ is H or C₁₋₆ alkyl; each R₁ is independently selected from H, halo (e.g. fluoro or chloro), C₇₋₁₂arylalkyl, C₂₋₁₂ alkenyl; OC₁₋₁₂ alkyl, SC₁₋₁₂ alkyl, OC₂₋₁₂ alkenyl, C₁₋₁₂ alkyl group, a C₆₋₁₄ aryl group, —OC₁₋₁₀alkyl-O—C₁₋₁₀alkyl, —C₁₋₁₀alkyl-O—C₁₋₁₀alkyl, OAr², O(CH₂)_{q}Ar², SAr² or S(CH₂)_{q}Ar²; wherein Ar² is phenyl, optionally substituted with one or more of halo, trihalo methyl, C₁₋₁₀-alkoxy, or C₁₋₁₀ alkyl; p is 0 to 3; q is 1 to 3, preferably 1 n is 1 to 4; or a salt, ester, solvate, N-oxide, or prodrug thereof, e.g. a salt thereof; for use in the treatment or prevention of a fibrotic disease.

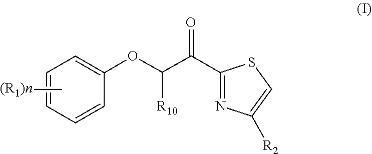

This invention relates to compositions for use in the treatment of fibrotic diseases or fibrotic disorders. In particular, the invention relates to the use of various 2-oxothiazole compounds in the treatment, prevention or reduction of symptoms of fibrosis or related conditions. The invention also relates to a method of treating, preventing, or reducing symptoms associated with a fibrotic disease or fibrotic disorder using the 2-oxothiazole compounds defined herein.

### BACKGROUND OF THE INVENTION

Fibrosis is an important cause of morbidity and mortality worldwide. Fibrosis is characterized by the accumulation of excess extracellular matrix components (e.g. collagen, fibronectin) that forms fibrous connective tissue in and around an inflamed or damaged tissue. Fibrosis may cause, for example, overgrowth, hardening, and/or scarring that disrupts the architecture of the underlying organ or tissue. While controlled tissue remodeling and scarring is part of the normal wound healing process, excessive and persistent scarring due to severe or repetitive injury or dysregulated wound healing can eventually lead to permanent scarring, organ dysfunction and failure, and even death.

There have been reports that fibrosis may be the result of a chronic inflammatory response after injury. Some common features of fibrotic diseases are excessive deposition of extracellular matrix (ECM) constituents, hardening, scarring and increased tension of the affected tissues [Gerarduzzi and Battista, 2017]. While controlled tissue remodeling and scarring is part of the normal wound healing process, excessive and persistent scarring due to severe or repetitive injury or dysregulated wound healing can eventually lead to permanent scarring, organ dysfunction and failure, and even death.

Fibrosis and related changes can occur in vascular disorders such as peripheral vascular disease, cardiac disease, cerebral disease and in all main tissue and organ systems (e.g., lung, liver, kidney, heart, skin). Fibrotic disorders include a wide range of clinical presentations, including multisystemic disorders, such as systemic sclerosis, multifocal fibrosclerosis, and organ-specific disorders, such as pulmonary, liver, and kidney fibrosis. While the etiology and causative mechanisms of individual fibrotic disorders may vary (e.g., ischemic event, exposure to a chemical, radiation, or infectious agent) and are not completely understood, nearly all share the common feature of abnormal and excessive deposition of extracellular matrix in affected tissues.

Transforming growth factor β1 (TGF-β1) is said to be a major driver of fibrosis, and induces the differentiation of fibroblasts to myofibroblasts. The differentiation into myofibroblasts is often characterized by de novo synthesis of α-smooth muscle actin (α-SMA).

There are reports that cPLA2α is a key player regulating arachidonic acid (AA) release for eicosanoid biosynthesis [Hao, 2007]. The cyclooxygenase (COX) enzymatic pathway converts AA to the biologically active, proinflammatory, eicosanoid prostaglandin E2 (PGE2).

The present inventors sought new methods for treating fibrotic diseases as there are no clearly effective therapies for treating, preventing, or reducing symptoms of fibrotic diseases. Thus, there is a need for effective methods of treating, preventing or reducing symptoms associated with these disorders. The present inventors have surprisingly found that certain 2-oxothiazoles can be used to treat, prevent or reduce symptoms of fibrotic diseases.

Various 2-oxothiazoles have been reported (see WO2011/039365, WO2014/118195, WO2016/016472). These compounds have not however, been previously suggested for use in the treatment, prevention or reduction of symptoms of fibrotic disorders.

### SUMMARY OF THE INVENTION

Thus, viewed from one aspect the invention provides a compound of formula (I)

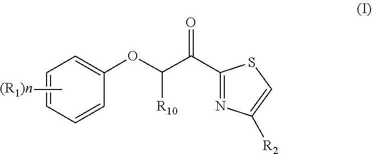

wherein R¹⁰ is H or C₁₋₆ alkyl such as Me;

R₂ is H, —(CH₂)ₚCOOH, —(CH₂)ₚCON(R⁵)₂ or —(CH₂)ₚCOOC₁₋₆alkyl;

each R⁵ is H or C₁₋₆ alkyl;

each R₁ is independently selected from H, halo (e.g. fluoro or chloro), C₇₋₁₂arylalkyl, C₂₋₁₂ alkenyl; OC₁₋₁₂ alkyl, SC₁₋₁₂ alkyl, OC₂₋₁₂ alkenyl, C₁₋₁₂ alkyl group, a C₆₋₁₄ aryl group, —OC₁₋₁₀alkyl-O—C₁₋₁₀alkyl, —C₁₋₁₀alkyl-O—C₁₋₁₀alkyl, OAr², O(CH₂)_{q}Ar², SAr² or S(CH₂)_{q}Ar²;

wherein Ar² is phenyl, optionally substituted with one or more of halo, trihalomethyl, C₁₋₁₀-alkoxy, or C₁₋₁₀ alkyl;

p is 0 to 3;

q is 1 to 3, preferably 1

n is 1 to 4;

or a salt, ester, solvate, N-oxide, or prodrug thereof, e.g. a salt thereof;

for use in the treatment or prevention of a fibrotic disease.

Viewed from another aspect the invention provides a method of treating, preventing or reducing symptoms associated with a fibrotic disease comprising administering to an animal, preferably a mammal, e.g. human, in need thereof, an effective amount of a compound of formula (I) or a salt, ester, solvate, N-oxide, or prodrug thereof as hereinbefore described.

Viewed from another aspect the invention provides use of a compound of formula (I) or a salt, ester, solvate, N-oxide, or prodrug thereof as hereinbefore described for use in the manufacture of a medicament for treating, preventing or reducing symptoms of a fibrotic disorder.

Viewed from another aspect the invention provides a method for reducing one or more of hydroxyproline content or collagen Type 1 mRNA expression in an organ in which the method includes the step of administering to an animal, preferably a mammal, e.g., human, in need thereof, an effective amount of a compound of formula (I) or a salt, ester, solvate, N-oxide, or prodrug thereof as hereinbefore described. In one embodiment of the method, the organ is selected from kidney, lung or liver.

Viewed from another aspect the invention provides use of a compound of formula (I) or a salt, ester, solvate, N-oxide, or prodrug thereof as hereinbefore described for use in the manufacture of a medicament for reducing one or more of hydroxyproline content or collagen Type I mRNA expression in an organ such as kidney, lung or liver.

### BRIEF DESCRIPTION OF THE FIGURES

FIGS. 1a-e shows that a 2-oxothiazole PLA2α inhibitor reduces the mRNA expression of certain fibrotic markers in TGF-β1 treated cells.

FIG. 2 shows that

FIG. 3 shows a box-plot representation showing a 95% confidence interval, mean and variance. Significance is calculated using the Welch's t-test for unequal variances or sample size t-test (n=4-8 independent experiments).

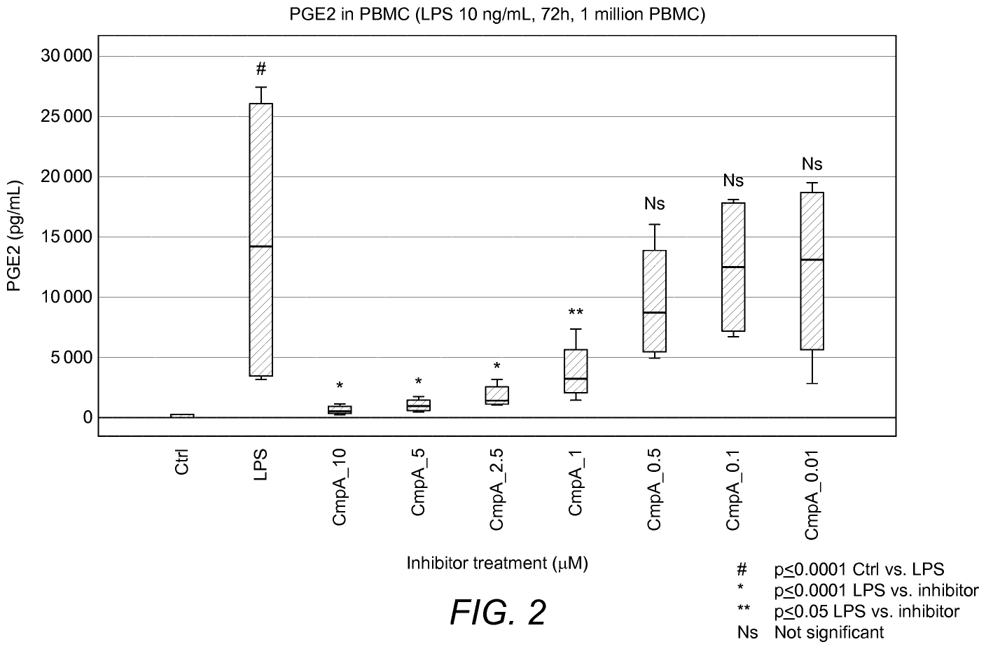

### DETAILED DESCRIPTION OF THE INVENTION

The instant disclosure provides methods for preventing, ameliorating, treating, or even reducing symptoms of a fibrotic disorder or fibrotic disease. As used herein the term “treating or preventing” may be understood to relate to treating or preventing the disease itself or treating or preventing symptoms associated with the disease, including reduction in the disease and/or symptoms and/or slowing the progression of the disease and/or symptoms. The term fibrosis describes the development of fibrous connective tissue as a reparative response to injury or damage. Repair of damaged tissues is a fundamental biological process that allows the ordered replacement of dead or injured cells during an inflammatory response, a mechanism that is crucial for survival. The repair process typically involves two distinct stages: a regenerative phase, in which injured cells are replaced by cells of the same type and there is no lasting evidence of damage; and a phase known as fibroplasia or fibrosis, in which connective tissue replaces normal parenchymal tissue. In most cases, both stages are required to slow or reverse the damage caused by an injurious agent. However, although initially beneficial, the healing process can become pathogenic if it continues unchecked, leading to considerable tissue remodelling and the formation of permanent scar tissue. Fibrotic scarring is often defined as a wound-healing response that has gone awry.

An injury is an event that damages tissue and initiates the wound healing process. After injury, both mechanical (i.e. extracellular stress caused by disruption of the extracellular matrix, ECM) and chemical signals (e.g. inflammatory mediators like TGF.beta.) activate fibroblastic cells to increase production of extra cellular matrix (ECM) components, which begins the process of fibroblast differentiation into myofibroblasts (Tomasek et al., Nat. Rev. Mol. Cell Biol. 3:349, 2002; Werner et al., Physiol. Rev. 83:835, 2003). Depending on the type of tissue being remodeled, the fibroblasts that differentiate may come from different sources, including locally present fibroblasts, pericytes, smooth muscle cells, fibrocytes from bone marrow, and from epithelial-mesenchymal transition (EMT) (Hinz et al., Am. J. Pathol. 170:1807, 2007). Wound healing is seen as complete when the newly formed, crosslinked ECM takes over the mechanical load, which is a signal to myofibroblasts to undergo apoptosis (Tomasek et al., 2002; Carlson et al., J. Surg. Res. 110:304, 2003).

If the injury is severe or repetitive, or if the wound-healing process is dysregulated, fibrosis becomes pathogenic, resulting in permanent scarring or hardening of the tissue, organ malfunction or failure, and ultimately death. For example, idiopathic pulmonary fibrosis (IPF) is not completely understood but is seen as a progressive and fatal lung disease that has few effective treatments other than lung transplantation (Mason et al., Ann. Thorac. Surg. 84:1121-8, 2007). Median survival of five years after diagnosis is less than 20%. Most forms of interstitial lung diseases and other forms of pulmonary fibrosis are characterized by fibrotic lesions, progressive distortion of alveolar architecture occurs and replacement with fibrotic or scar tissues with excess ECM deposition (American Thoracic Society, Am. J. Respir. Crit. Care Med. 161:646, 2000; Noble et al., Clin. Chest Med. 25:749, 2004; Selman et al., Ann. Intern, Med. 134:136, 2001). This can result in progressive dyspnea and loss of lung function. A hallmark morphological lesion is spatial and temporal heterogeneity incorporating areas of normal lung being directly adjacent to areas of fully established fibrosis, microscopic honeycombing, and areas of evolving fibrosis containing collagen-producing fibroblasts/myofibroblasts, often referred to as “fibrotic foci.”

The term “fibrotic disorder” or “fibrotic disease” (which are used interchangeably herein) refers to a medical condition featuring progressive and/or irreversible fibrosis, wherein excessive deposition of extracellular matrix occurs in and around inflamed or damaged tissue. In certain embodiments, a fibrotic disorder or disease is associated with the persistent presence of myofibroblasts in and around fibrotic foci or lesions. Excessive and persistent fibrosis can progressively remodel and destroy normal tissue, which may lead to dysfunction and failure of affected organs, and ultimately death. A fibrotic disorder may affect any tissue in the body and is generally initiated by an injury and the transdifferentiation of fibroblasts into myofibroblasts.

As used herein, “injury” refers to an event that damages tissue and initiates fibrosis. An injury may be caused by an external factor, such as mechanical insult (e.g., cut, surgery), exposure to radiation, chemicals (e.g., chemotherapy, toxins, irritants, smoke), or infectious agent (e.g., bacteria, virus, or parasite). An injury may be caused by, for example, chronic autoimmune inflammation, allergic response, HLA mismatching (e.g., transplant recipients), or ischemia (i.e., an “ischemic event” or “ischemia” refers to an injury that restricts in blood supply to a tissue, resulting in damage to or dysfunction of tissue, which may be caused by problems with blood vessels, atherosclerosis, thrombosis or embolism, and may affect a variety of tissues and organs; an ischemic event may include, for example, a myocardial infarction, stroke, organ or tissue transplant, or renal artery stenosis). In certain embodiments, an injury leading to a fibrotic disorder may be of unknown etiology (i.e., idiopathic).

Non-limiting examples of fibrotic disorders or fibrotic diseases include renal (kidney) fibrosis, pulmonary fibrosis, such as idiopathic pulmonary fibrosis, cystic fibrosis, liver fibrosis (e.g., cirrhosis), cardiac fibrosis, endomyocardial fibrosis, vascular fibrosis (e.g., atherosclerosis, stenosis, restenosis), atrial fibrosis, mediastinal fibrosis, myelofibrosis, retroperitoneal fibrosis, progressive massive fibrosis (e.g., lungs), nephrogenic systemic fibrosis, Crohn's disease, hypertrophic scarring, keloid, scleroderma, systemic sclerosis (e.g., skin, lungs), athrofibrosis (e.g., knee, shoulder, other joints), Peyronie's disease, Dupuytren's contracture, adhesive capsulitis, organ transplant associated fibrosis, ischemia associated fibrosis, or the like.

In one embodiment, the invention relates to the treatment, prevention or reduction of symptoms associated with renal (kidney) fibrosis. Reference to “renal fibrosis” or “kidney fibrosis” (the two terms are used interchangeably herein) means a progressive fibrotic manifestation of various diseases which may lead to severe illness and even death. For example, chronic kidney disease (CKD) can present in some patients with a potentially life-threatening fibrotic phenotype. This condition (CKD with fibrosis) may result from a variety of other serious indications such as diabetic nephropathy, hypertension, glomerulonephritis (GN) and polycystic disease. Without wishing to be bound by theory, it is believed that prior therapies intended to treat these diseases are often inadequate to halt the development or progression of fibrosis leading to severe illness and death in some cases. The present invention addresses this problem, for example, by providing a method that focuses on the development and/or progression of fibrosis in a patient that has, is suspected of having or is at risk of developing CKD, diabetic neuropathy, hypertension, glomerulonephritis (GN) and/or a polycystic disease.

In another embodiment, the invention relates to the treatment of a pulmonary fibrotic disorder. Reference to “pulmonary fibrotic disorder” means diseases or disorders characterized by fibrotic hypertrophy or fibrosis of lung tissue. Exemplary pulmonary fibrotic disorders include pulmonary fibrosis, idiopathic pulmonary fibrosis, interstitial lung disease, interstitial pulmonary fibrosis, chronic interstitial pneumonitis, Hamman-Rich Syndrome, usual interstitial pneumonitis (UIP), fibrosing alveolitis, pulmonary sarcoidosis, progressive massive fibrosis (e.g., lungs), systemic sclerosis (e.g., lungs), lung transplant associated fibrosis, or the like.

This invention involves the use of compounds of formula (I) or salts, ester, solvate, N-oxide, or prodrug thereof in the treatment or prevention of fibrotic disorders. In a first embodiment, the invention provides 2-oxothiazole compounds of formula (I)

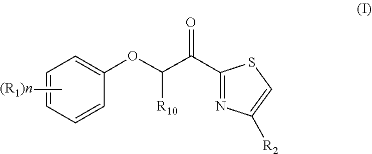

as hereinbefore defined or a salt, ester, solvate, N-oxide, or prodrug thereof for use in the treatment or prevention of a fibrotic disease.

In compounds of formula (I) it is preferred if R₂ is not H.

It is preferred if R₂ is —(CH₂)ₚCOOH, —(CH₂)ₚCONHMe, —(CH₂)ₚCONH₂ or —(CH₂)ₚCOOC₁₋₆alkyl. Most especially, R₂ is —COOCH₃ or —COOCH₂CH₃.

The subscript p is preferably 0 or 1, especially 0. A most preferred option for R₂ is —COOC₁₋₆alkyl, especially —COOC₁₋₂alkyl.

R₁₀ is preferably methyl or H.

In compounds of formula (I) there are preferably 0, 1 or 2 groups R¹, especially 1, i.e. n is preferably 1.

If there is one substituent R₁ on the Ph ring, it is preferred if it is para to the O atom. If there are two substituents, it is preferred if the substituents are positioned on adjacent carbon atoms, ideally the meta and para positions on the ring.

Preferred options for R₁ are a C₁₋₁₀alkyl group, a —OC₁₋₁₀ alkyl group, —SC₁₋₁₀ alkyl or OAr² group.

More preferred options include C₄₋₁₀alkyl group, a —OC₄₋₁₀ alkyl group, —SC₄₋₁₀ alkyl or OAr² group.

Any R₁ alkyl groups are preferably linear. Linear alkyl groups are also preferred in alkyl groups that form part of other substituents such as alkyl groups within alkoxy, S-alkyl groups and so on.

Ar² is preferably phenyl or phenyl substituted with halo, e.g. F. That substituent is preferably in the para position.

In a preferred embodiment therefore, compounds of use in the invention are of formula (II):

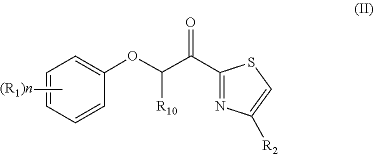

wherein R¹⁰ is H or Me;

R₂ is —(CH₂)ₚCON(R⁵)₂ or —(CH₂)ₚCOOC₁₋₆alkyl;

each R⁵ is H or C₁₋₆ alkyl;

R₁ is —OC₁₋₁₂ alkyl, —SC₁₋₁₂ alkyl, C₁₋₁₂ alkyl group, OAr², O(CH₂)_{q}Ar², SAr² or S(CH₂)_{q}Ar²;

wherein Ar² is phenyl, optionally substituted with one or more of halo, trihalomethyl, C₁₋₁₀-alkoxy, or C₁₋₁₀ alkyl;

p is 0 to 1;

q is 1;

n is 1;

or a salt, ester, solvate, N-oxide, or prodrug thereof, e.g. a salt thereof.

In a more preferred embodiment, compounds of use in the invention are of formula (III):

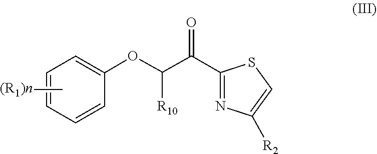

wherein R¹⁰ is H or Me;

R₂ is —(CH₂)ₚCON(R⁵)₂ or —(CH₂)ₚCOOC₁₋₆alkyl;

each R⁵ is H or Me;

R₁ is —OC₁₋₁₂ alkyl, —SC₁₋₁₂ alkyl, C₁₋₁₂ alkyl group, or OAr²;

wherein Ar² is phenyl, optionally substituted with halo;

p is 0 to 1;

n is 1;

or a salt, ester, solvate, N-oxide, or prodrug thereof, e.g. a salt thereof.

In an even more preferred embodiment, compounds of use in the invention are of formula (IV):

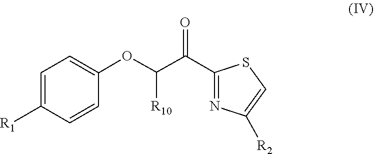

wherein R¹⁰ is H or Me;

R₂ is CONHR⁵ or COOC₁₋₆alkyl;

R⁵ is H or Me;

R₁ is —OC₁₋₁₂ alkyl, —SC₁₋₁₂ alkyl, C₁₋₁₂ alkyl group, or OAr²;

wherein Ar² is phenyl, optionally substituted with halo;

or a salt, ester, solvate, N-oxide, or prodrug thereof, e.g. a salt thereof.

In a most preferred embodiment, compounds of use in the invention are of formula (V):

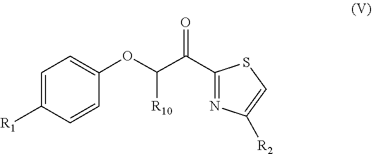

wherein R¹⁰ is H or Me;

R₂ is CONHR⁵ or COOC₁₋₂alkyl;

R⁵ is H or Me;

R₁ is —OC₄₋₁₀ alkyl, —SC₄₋₁₀ alkyl, C₄₋₁₀ alkyl group, or OAr²;

wherein Ar² is phenyl, optionally substituted with halo;

or a salt, ester, solvate, N-oxide, or prodrug thereof, e.g. a salt thereof.

In a most preferred embodiment, compounds of use in the invention are of formula (VI):

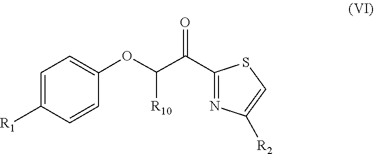

wherein R¹⁰ is H or Me;

R₂ is COOC₁₋₂alkyl;

R₁ is —OC₄₋₁₀ alkyl, —SC₄₋₁₀ alkyl, C₄₋₁₀ alkyl group, or OAr²;

wherein Ar² is phenyl, optionally substituted with halo;

or a salt, ester, solvate, N-oxide, or prodrug thereof, e.g. a salt thereof.

Highly preferred compounds for use in the invention are depicted below.

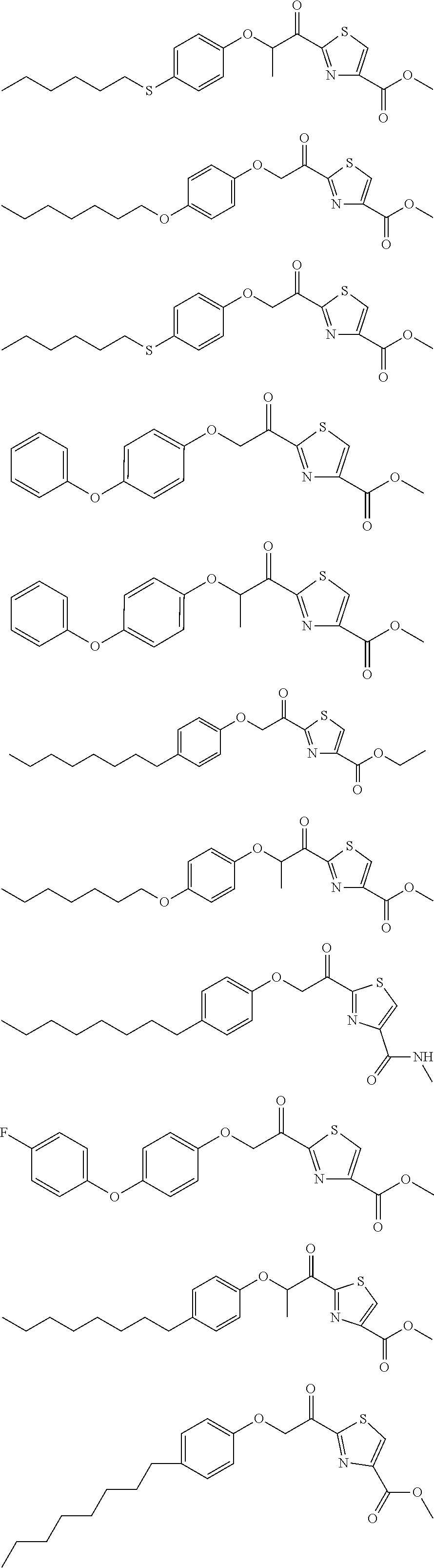

Most preferred compounds are:

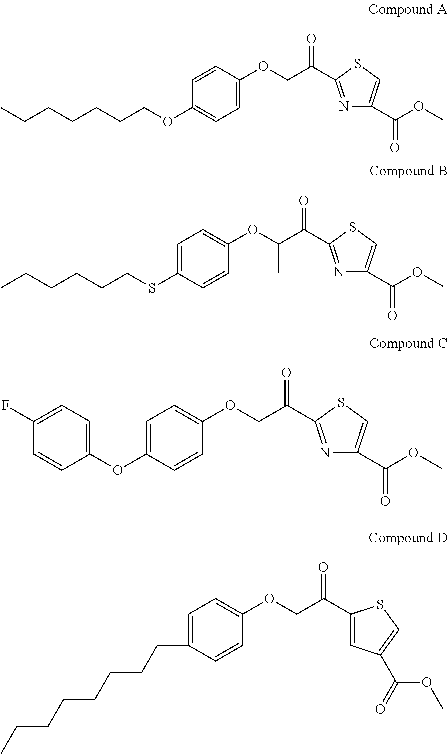

Where possible, the compounds of the invention can be administered in salt, hydrate or solvate form, especially salt form.

Typically, a pharmaceutical acceptable salt may be readily prepared by using a desired acid. The salt may precipitate from solution and be collected by filtration or may be recovered by evaporation of the solvent. For example, an aqueous solution of an acid such as hydrochloric acid may be added to an aqueous suspension of a compound of formula (I) and the resulting mixture evaporated to dryness (lyophilised) to obtain the acid addition salt as a solid. Alternatively, a compound of formula (I) may be dissolved in a suitable solvent and the acid may be added in the same solvent or another suitable solvent. The resulting acid addition salt may then be precipitated directly, or by addition of a less polar solvent such as diisopropyl ether or hexane, and isolated by filtration.

Suitable addition salts are formed from inorganic or organic acids which form non-toxic salts and examples are hydrochloride, hydrobromide, hydroiodide, sulphate, bisulphate, nitrate, phosphate, hydrogen phosphate, acetate, trifluoroacetate, maleate, malate, fumarate, lactate, tartrate, citrate, formate, gluconate, succinate, pyruvate, oxalate, oxaloacetate, trifluoroacetate, saccharate, benzoate, alkyl or aryl sulphonates (e.g. methanesulphonate, ethanesulphonate, benzenesulphonate or p-toluenesulphonate) and isethionate. Representative examples include trifluoroacetate and formate salts, for example the bis or tris trifluoroacetate salts and the mono or diformate salts, in particular the tris or bis trifluoroacetate salt and the monoformate salt.

Compounds of formula (I) may be manufactured using known chemical synthetic routes. The manufacture of the compounds of the invention typically involves known literature reactions. Variations of the substituents on the heterocyclic rings and manipulation of the side chain binding the carbonyl can be achieved using all manner of synthetic techniques which the skilled man will know. In particular, reference is made to WO2011/039365, WO2014/118195, and WO2016/016472 which all describe synthetic pathways to compounds of this invention. Compounds of the invention can therefore be prepared following the teaching in these references.

The amount of the compounds of the invention required in a dosage form will often be determined by the physician.

The composition of the invention is proposed primarily for use in the treatment or prevention of fibrotic disorders.

By treating or treatment is meant at least one of:

(i) inhibiting the disease i.e. arresting, reducing or delaying the development of the disease or a relapse thereof or at least one clinical or subclinical symptom thereof, or
(ii) relieving or attenuating one or more of the clinical or subclinical symptoms of the disease.

By prevention is meant (i) preventing or delaying the appearance of clinical symptoms of the disease developing in a mammal.

The benefit to a subject to be treated is either statistically significant or at least perceptible to the patient or to the physician. In general a skilled man can appreciate when “treatment” occurs. It is particularly preferred if the composition of the invention are used therapeutically, i.e. to treat a condition which has manifested rather than prophylactically. It may be that the composition of the invention is more effective when used therapeutically than prophylactically.

The composition of the invention can be used on any animal subject, in particular a mammal and more particularly to a human or an animal serving as a model for a disease (e.g., mouse, monkey, etc.).

In order to treat a disease an effective amount of the active composition needs to be administered to a patient. A “therapeutically effective amount” means the amount of a composition that, when administered to an animal for treating a state, disorder or condition, is sufficient to effect such treatment. The “therapeutically effective amount” will vary depending on the composition, the disease and its severity and the age, weight, physical condition and responsiveness of the subject to be treated and will be ultimately at the discretion of the attendant doctor.

It may be that to treat fibrosis according to the invention that the composition of the invention has to be re-administered at certain intervals. Suitable dosage regimes can be prescribed by a physician.

The composition of the invention typically comprises the active components in admixture with at least one pharmaceutically acceptable carrier selected with regard to the intended route of administration and standard pharmaceutical practice.

The term “carrier” refers to a diluent, excipient, and/or vehicle with which an active compound is administered. The pharmaceutical compositions of the invention may contain combinations of more than one carrier. Such pharmaceutical carriers are well known in the art. The pharmaceutical compositions may also comprise any suitable binder(s), lubricant(s), suspending agent(s), coating agent(s), and/or solubilizing agent(s) and so on. The compositions can also contain other active components, e.g. other drugs for the treatment of cancer.

It will be appreciated that pharmaceutical composition for use in accordance with the present invention may be in the form of oral, parenteral, transdermal, sublingual, topical, implant, nasal, or enterally administered (or other mucosally administered) suspensions, capsules or tablets, which may be formulated in conventional manner using one or more pharmaceutically acceptable carriers or excipients. The compositions of the invention could also be formulated as nanoparticle formulations.

However, for the treatment of fibrosis, the composition of the invention can be administered by a variety of routes such as orally or parenterally (e.g., subcutaneous, intramuscular or intravenous). For many invention embodiments subcutaneous administration will be preferred. In embodiments in which compositions of the invention are administered orally, the composition may therefore be provided in the form of a tablet or solution for injection.

The pharmaceutical composition of the invention may contain from 0.01 to 99% weight—per volume of the active material. The therapeutic doses will generally be between about 10 and 2000 mg/day and preferably between about 30 and 1500 mg/day. Other ranges may be used, including, for example, 50-500 mg/day, 50-300 mg/day, 100-200 mg/day.

Administration may be once a day, twice a day, or more often, and may be decreased during a maintenance phase of the disease or disorder, e.g. once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art.

Treatment according to the invention may be carried out in conjunction with other known treatments for the fibrotic disease in question. For example, patients with pulmonary fibrous may be administered oxygen. Treatment according to the invention may be carried out in other embodiments in conjunction with other known treatments in the same or different pharmaceutical compositions. Illustrative “combination agents” may include, among others: an immunosuppressive drug including, but not limited to, cyclosporine, azathioprine, cyclophosphamide, or mycophenolate mofetil; an anti-inflammatory drug including, but not limited to, corticosteroids (e.g. prednisone), a cytokine including but not limited to, interferon-alpha, interferon gamma, interleukin 12, a TNF-, CCR2-, CCR5- or VAP1-inhibitor; thalidomide; an anti-hypertensive agent including but not limited to ACE inhibitors, ARBs, renin inhibitors and mineralcorticoid receptor antagonists (e.g. captopril, ramipril, lisinopril, losartan, telmisartan, aliskiren, spironolactone, finerenone, CS-3150, MT-3995, eplerenone etc); a monoclonal antibody or other agent targeting, among others, CTGF, TGF-β, MCP-1, IL-4, and IL-13; a multiple receptor tyrosine kinase inhibitor including, but not limited to, nintedanib and the JNK (kinase) inhibitor tanzisertib (CC-930) or ruxolitinib (Jakavi™); an antioxidant such as, but not limited to, N-acetylcysteine, pirfenidone, vitamin E, S-adenosyl methionine, pyridorin, GKT137831, or penicillamine; an enzyme inhibitor including, but not limited, to Lysyloxidase-like-2 (LOXL2 enzyme); an integrin inhibitor such as, but not limited to, αᵥβ₆; a lipid receptor modulator or a hypolipidemic agent including, but not limited to, lysophosphatidic acid receptor antagonists, HMG-CoA reductase inhibitors, Stearoyl-CoA Desaturase inhibitors, PPAR inhibitors and thiazolindiones or cholesterol absorption inhibitors; an inhibitor affecting vasculature or angiogenesis including but not limited to ET_{A} inhibitors such as atrasentan, SGLT2 inhibitors such as canagliflozin; pomalidomide; an apoptosis inhibitor such as IDN-6556 or GS-4997; a PDE inhibitor such as CTP-499; a thrombomodulin inhibitor or a therapy based on stem cells (SCs) such as umbilical cord SCs, autologous SCs and bone marrow SCs;

The invention is described further below with reference to the following non-limiting examples and figures.

### BRIEF DESCRIPTION OF THE FIGURES

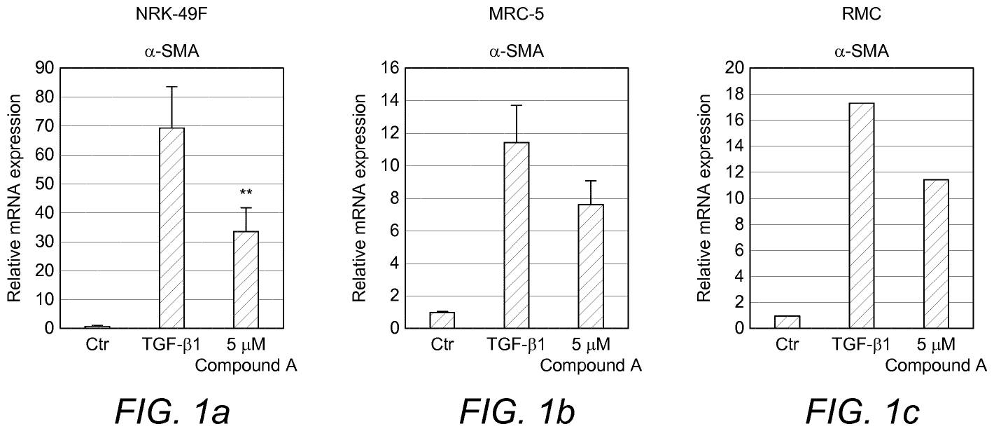

FIG. 1a-e shows that cPLA2α inhibitor Compound A inhibits TGF-β1 induced expression of α-SMA mRNA in NRK-49F cells. FIG. 1 also shows that in the mesangial cell line, RMC, and Compound A seems to reduce α-SMA mRNA expression. In MRC-5 cells, FIG. 1 shows that Compound A reduced Ptgs2 expression and Ptgs2 mRNA levels.

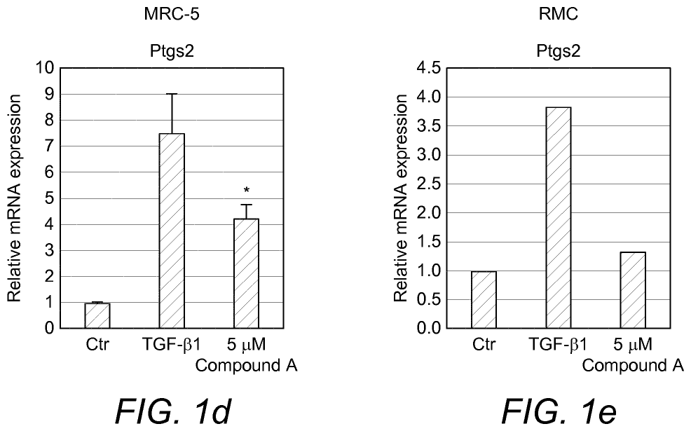

FIG. 2 shows the dose-dependent inhibition of PGE2 in human PBMC.

### Example 1

This example uses:

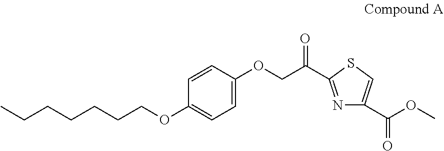 [SMILES: CCCCCCCOc1ccc(OCC(=O)c2nc(C(=O)OC)cs2)cc1]

The preparation of this compound is described in WO2014/118195.

The Following Materials and Methods were Used as Needed.

### Cell Culture

NRK-49F (normal rat kidney fibroblasts, ATCC® CRL-1570™) were maintained in DMEM with 4500 mg/L glucose, 5% FBS, L-glutamine, and gentamicin. For experiments, 3×10\hat{ }5 cells/well were seeded in 6-well plates. After three days, post-confluent cells were serum starved for 24 h. They were then pre-incubated with inhibitors for 90 min, before they were treated with 10 ng/ml TGF-β1 for 24 h (mRNA-expression).

MRC-5 (human lung fibroblast, ATCC® CCL-171™) were maintained in MEM with 10% FBS, L-glutamine, and gentamicin. For experiments, 1×10\hat{ }5 cells/well were seeded in 6-well plates. After two days, pre-confluent cells were serum starved for 24 h. They were then pre-incubated with inhibitors for 90 min, before they were treated with 2 ng/ml TGF-β1 for 24 h.

RMC (rat mesangial cells, ATCC® CRL-2573™) were maintained in DMEM with 4500 mg/L glucose (Sigma), 15% FBS, L-glutamine, and 0.4 mg/ml G418. For experiments, 3×10\hat{ }5 cells/well were seeded in 6-well plates. After five days, post-confluent cells were serum starved for 24 h. They were then pre-incubated with inhibitors for 90 min, before they were treated with 5 ng/ml TGF-β1 for 24 h.

qRT-PCR

Total RNA was isolated using the RNeasy Mini Kit (Qiagen). cDNA synthesis was performed with 1 μg total RNA in 20 μl reaction of QuantiTect Reverse Transcription Kit (Qiagen). After synthesis, cDNA was diluted 1:6 with RNase-free water. qRT-PCR was performed with LightCycler 480 SYBR Green I Master (Roche), and the qRT-PCR analyses were carried out using the Lightcycler 96 system (Roche). Primer sequences are listed in Table 1.

TABLE 1
Primer sequences

| Oligo | | |
|---|---|---|
| name | Forward (5′-3′) | Reverse (5′-3′) |
| Rat | GCCATCAGGAACCTCGAGAA | GGAGCATCATCACCAGCAAAG |
| Acta2 | | |
| Rat | CTCATACTGATAGGAGAGACG | TCGAACTTGAGTTTGAAGTG |
| Ptgs2 | | |
| Rat | CGATTCCGTGGGTGGTGGTG | CATGCCAGAGTCTCGTTCGT |
| 18S | | TATC |
| Human | AGATCAAGATCATTGCCCC | TTCATCGTATTCCTGTTTGC |
| ACTA2 | | |
| Human | AAGCAGGCTAATACTGATAGG | TGTTGAAAAGTAGTTCTGGG |
| PTGS2 | | |
| Human | CAGAAGGATGTAAAGGATGG | TATTTCTTCTTGGACACACC |
| RPS18 | | |

The results show that a 2-oxothiazole (Compound A) PLA2α inhibitor reduces the mRNA expression of certain fibrotic markers in TGF-β1 treated cells. The data show, among other things, that a 2-oxothiazole reduced pathological fibrotic processes.

As shown in FIG. 1 a-c, TGF-β1 is a powerful inducer of α-SMA mRNA in both fibroblast cell lines tested and in the mesangial cell line. The cPLA2α inhibitor Compound A (5 μM) inhibits TGF-β1 induced expression of α-SMA mRNA in NRK-49F cells by 50% (FIG. 1a). Also in the mesangial cell line, RMC, and Compound A seems to reduce α-SMA mRNA expression around 35%, however not significantly (FIG. 1c).

TGF-β1 induces mRNA expression of Ptgs2 (encoding the COX2 protein) in both cell lines tested. In MRC-5 cells, 5 μM Compound A reduced Ptgs2 expression (FIG. 1d). Also in RMC, 5 μM Compound A seem to reduce Ptgs2 mRNA levels (FIG. 1e).

Abbreviations: TGF-β1, transforming growth factor β1; α-SMA, α-smooth muscle actin; Ptgs2, prostaglandin endoperoxide synthase 2/cyclooxygenase 2; Col1a2, collagen 1a2; Col3a1, collagen 3a1; Col4a1, collagen 4a1; CTGF, connective tissue growth factor.

### Example 2

### PBMC Isolation and Treatment

Blood was recruited from healthy donors at St. Olavs Hospital HF, the Bloodbank (project approved by Regional Ethical Committee of Mid-Norway; #2016/553). Peripheral blood mononuclear cells (PBMC) were isolated using SepMate™ separation tubes with LymphoPrep density gradient medium according to the STEMCELL Technology recommendations. For experiments, 1×10′6 cells/well/1 mL RPMI medium supplemented with 5% FBS, 0.3 mg/ml glutamine and 0.1 mg/ml gentamicin with- or without inhibitor. Treatments were added 2 hours prior to the addition of lipopolysaccharides as a potent inducer of inflammation (LPS from E. coli 026:B6 γ-irradiated, Sigma-Aldrich, #L2654). Following treatment (72 hrs., 37° C., 5% CO2), the cell suspensions were centrifuged to isolate the supernatant from the cell fraction. Samples were stored at −80° C. until analysis.

### Enzyme Linked Immunoassay Detection of PGE2

PBMC supernatants were analyzed by enzyme-linked immunosorbent assay (EIA) for PGE2 (Cayman #514010), according to the manufacturers protocols. PBMC supernatants were assayed at dilutions of 1:100, except supernatants from non-LPS treated PBMC that were assayed undiluted in all assays. Supernatants were hybridized over-night incubation, enzymatic conversion of substrate were read at OD420 nm. Data were processed using a 4-parameter logistic fit model.

Compound A inhibits PGE2 production in human PBMC in a dose dependent manner.

To assess the anti-inflammatory effect of Compound A in a human model system, we isolated peripheral blood mononuclear cells (PBMC) from healthy blood donors. In response to LPS (10 ng/mL), PGE2 production increased from 135+/−87 pg/mL to 18185+/−11200 pg/mL. In response to Compound A, PGE2 production was reduced to 510+/−406 pg/mL in the highest dose applied (10 μM), and the inhibition was clearly dose-dependent with an estimated IC50 of 0.44 μM (FIG. 2). Furthermore, both PGE2 induction and inhibition were highly significant as given by Welch's t-test for unequal variances or sample size (n=4-8 independent experiments).

Results are presented in FIG. 2. FIG. 2 shows the dose-dependent inhibition of PGE2 in human PBMC. Bpx-plot representation showing a 95% confidence interval, mean and variance. Significance is calculated using the Welch's t-test for unequal variances or sample size t-test (n=4-8 independent experiments).

The reduction of PGE2 in PBMC indicate that Compound A is a potent anti-inflammatory compound.

### Example 3

### Rat Renal Mesangial Cell Culture and Treatment

Cell culture of rat renal mesangial cells were isolated, characterized and cultured as previously described (Pfeilschifter eteil., e1984).84ERLINK “htmesangial cells in 24-well plates were pretreated for 90library.wiley.com/doi/full/10.1111/Compound A before stimulation in a volume of 0.5 mL with IL-1eforenM, Cell Concept GmbH) to induce PGE2Hn in a volume of 0.5 mL with ILcom/doi/full⁰C, 5% CO2), the cell suspensions were centrifuged to isolate the supernatant from the cell fraction. Samples were stored at −80° C. until analysis.

Rat mesangial cell supernatants were analyzed by EIA for PGE2 (Assay Designs, BIOTREND Chemikalien GmbH), according to the manufacturers protocols. Data were calculated as pg of PGE₂ per 1.3×10⁵ cells which was the cell number per well. Data shows 65% inhibition of PGE2 at 10 μM and an estimated IC50 of 0.9 μM.

### REFERENCES

Halper J., Kjaer M. (2014), Progress in Heritable Soft Connective Tissue Diseases. Advances in Experimental Medicine and Biology, vol 802. Springer, Dordrecht
Gerarduzzi, C., Di Battista, J. A. Inflamm. Res. (2017) 66: 451.
Rayego-Mateos, S., Morgado-Pascual, J. L., Rodrigues-Diez, R. R., Rodrigues-Diez, R., Falke, L. L., Mezzano, S., Ortiz, A., Egido, J., Goldschmeding, R. and Ruiz-Ortega, M. (2018), J. Pathol, 244: 227-241. doi:10.1002/path.5007
Hao C. M., Breyer M. D, (2007), Semin Nepbrol 27:338-351

What is claimed is:

1. A compound of formula (I) 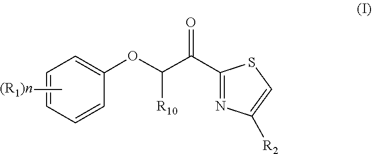
wherein R¹⁰ is H or C₁₋₆ alkyl such as Me;
R₂ is H, —(CH₂)ₚCOOH, —(CH₂)ₚCON(R⁵)₂ or —(CH₂)ₚCOOC₁₋₆alkyl;
each R⁵ is H or C₁₋₆ alkyl;
each R₁ is independently selected from H, halo (e.g. fluoro or chloro), C₇₋₁₂arylalkyl, C₂₋₁₂ alkenyl; OC₁₋₁₂ alkyl, SC₁₋₁₂ alkyl, OC₂₋₁₂ alkenyl, C₁₋₁₂ alkyl group, a C₆₋₁₄ aryl group, —OC₁₋₁₀alkyl-O—C₁₋₁₀alkyl, —C₁₋₁₀alkyl-O—C₁₋₁₀alkyl, OAr², O(CH₂)_{q}Ar², SAr² or S(CH₂)_{q}Ar²;
wherein Ar² is phenyl, optionally substituted with one or more of halo, trihalomethyl, C₁₋₁₀-alkoxy, or C₁₋₁₀ alkyl;
p is 0 to 3;
q is 1 to 3, preferably 1
n is 1 to 4;
or a salt, ester, solvate, N-oxide, or prodrug thereof, e.g. a salt thereof;
for use in the treatment or prevention of a fibrotic disease.

2. A compound as claimed in claim 1 wherein R₂ is —COOCH₃ or —COOCH₂CH₃.

3. A compound for use as claimed in any preceding claim wherein R₁₀ is methyl or H.

4. A compound for use as claimed in any preceding claim wherein n=1 and R₁ is in the para position.

5. A compound for use as claimed in any preceding claim wherein R₁ is a C₁₋₁₀alkyl group, a —OC₁₋₁₀ alkyl group, —SC₁₋₁₀alkyl or OAr² group.

6. A compound for use as claimed in claim 5 wherein any alkyl group in the groups C₁₋₁₀alkyl group, a —OC₁₋₁₀ alkyl group, or —SC₁₋₁₀alkyl is linear.

7. A compound for use as claimed in any preceding claim wherein R₁ is C₄₋₁₀alkyl group, a —OC₄₋₁₀ alkyl group, —SC₄₋₁₀alkyl or OAr² group, and Ar² is phenyl or phenyl substituted with halo, e.g. F, preferably in the para position.

8. A compound for use as claimed in any preceding claim of formula (II): 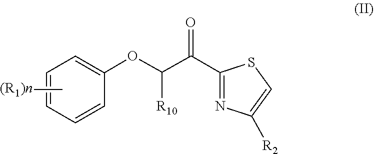
wherein R¹⁰ is H or Me;
R₂ is —(CH₂)ₚCON(R⁵)₂ or —(CH₂)ₚCOOC₁₋₆alkyl;
each R⁵ is H or C₁₋₆ alkyl;
R₁ is —OC₁₋₁₂ alkyl, —SC₁₋₁₂ alkyl, C₁₋₁₂ alkyl group, OAr², O(CH₂)_{q}Ar², SAr² or S(CH₂)_{q}Ar²;
wherein Ar² is phenyl, optionally substituted with one or more of halo, trihalomethyl, C₁₋₁₀-alkoxy, or C₁₋₁₀ alkyl;
p is 0 to 1;
q is 1;
n is 1;
or a salt, ester, solvate, N-oxide, or prodrug thereof, e.g. a salt thereof.

9. A compound for use as claimed in any preceding claim of formula (III): 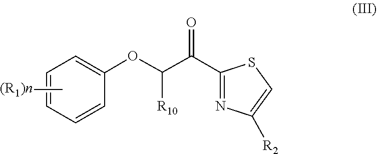
wherein R¹⁰ is H or Me;
R₂ is —(CH₂)ₚCON(R⁵)₂ or —(CH₂)ₚCOOC₁₋₆alkyl;
each R⁵ is H or Me;
R₁ is —OC₁₋₁₂ alkyl, —SC₁₋₁₂ alkyl, C₁₋₁₂ alkyl group, or OAr²;
wherein Ar² is phenyl, optionally substituted with halo;
p is 0 to 1;
n is 1;
or a salt, ester, solvate, N-oxide, or prodrug thereof, e.g. a salt thereof.

10. A compound for use as claimed in any preceding claim of formula (III): 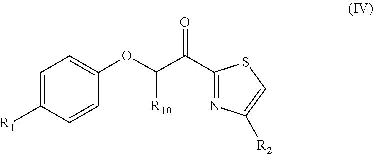
wherein R¹⁰ is H or Me;
R₂ is CONHR⁵ or COOC₁₋₆alkyl;
R⁵ is H or Me;
R₁ is —OC₁₋₁₂ alkyl, —SC₁₋₁₂ alkyl, C₁₋₁₂ alkyl group, or OAr²;
wherein Ar² is phenyl, optionally substituted with halo;
or a salt, ester, solvate, N-oxide, or prodrug thereof, e.g. a salt thereof.

11. A compound for use as claimed in any preceding claim of formula (V): 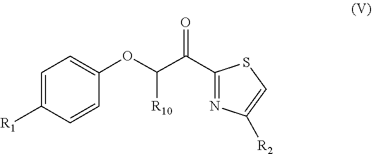
wherein R¹⁰ is H or Me;
R₂ is CONHR⁵ or COOC₁₋₂alkyl;
R⁵ is H or Me;
R₁ is —OC₄₋₁₀ alkyl, —SC₄₋₁₀ alkyl, C₄₋₁₀ alkyl group, or OAr²;
wherein Ar² is phenyl, optionally substituted with halo;
or a salt, ester, solvate, N-oxide, or prodrug thereof, e.g. a salt thereof.

12. A compound for use as claimed in any preceding claim of formula 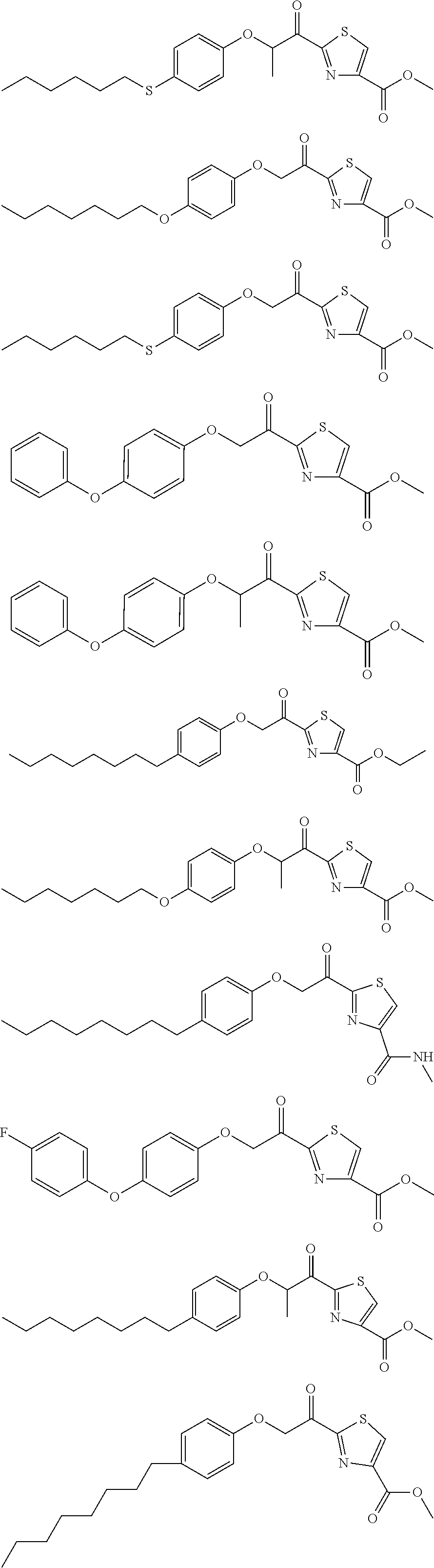
or a salt thereof.

13. A compound for use as claimed in any preceding claim of formula 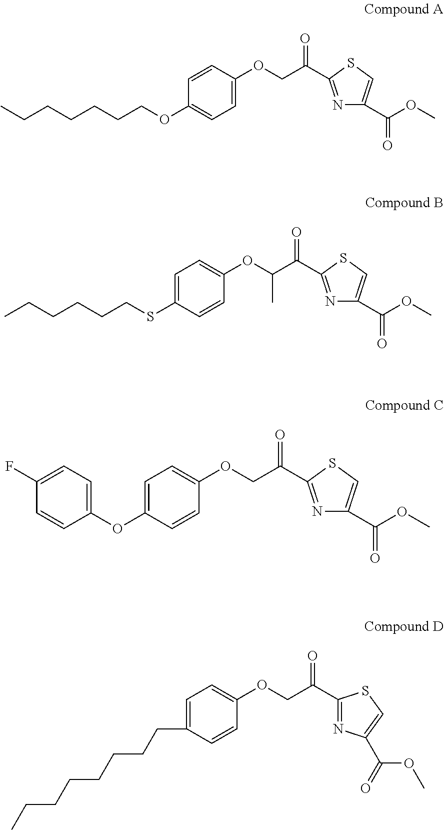
or a salt thereof.

14. A compound for use as claimed in any preceding claim wherein the fibrotic disease is due to injury or is idiopathic.

15. A compound for use as claimed in claim 14 wherein the injury is an ischemic event or due to exposure to radiation, a chemical, or an infectious agent.

16. A compound for use as claimed in any preceding claim wherein the compound is administered after a fibrotic lesion has developed in the subject.

17. A compound for use as claimed in any preceding claim wherein the compound is formulated with a pharmaceutically acceptable excipient.

18. A compound for use as claimed in any preceding claim wherein the compound is administered in combination with one or more adjunctive therapeutic agents.

19. A compound for use as claimed in any preceding claim wherein the fibrotic disease is selected from kidney fibrosis, pulmonary fibrosis, idiopathic pulmonary fibrosis, cystic fibrosis, liver fibrosis, cardiac fibrosis, endomyocardial fibrosis, atrial fibrosis, mediastinal fibrosis, myelofibrosis, retroperitoneal fibrosis, nephrogenic systemic fibrosis, Crohn's disease, hypertrophic scarring, keloid, scleroderma, organ transplant-associated fibrosis, or ischemia-associated fibrosis.

20. A compound for use as claimed in any preceding claim wherein the fibrotic disease is pulmonary or kidney fibrosis.

21. A compound for use as claimed in any preceding claim wherein the fibrotic disease is liver fibrosis.

22. A compound for use as claimed in any preceding claim wherein the fibrotic disease is chronic kidney disease or nephtrogenic systemic fibrosis.

23. A method of treating or preventing a fibrotic disease comprising administering to an animal, preferably a mammal, e.g. human, in need thereof, an effective amount of a compound of formula (I) or a salt, ester, solvate, N-oxide, or prodrug thereof, e.g. a salt thereof as defined in claim 1 to 13.

24. Use of a compound of formula (I) or a salt, ester, solvate, N-oxide, or prodrug thereof, e.g. a salt thereof as defined claim 1 to 13 for use in the manufacture of a medicament for treating or preventing a fibrotic disorder.

25. A method for reducing one or more of hydroxyproline content or collagen Type 1 mRNA expression in an organ, the method comprising administering to an animal, preferably a mammal, e.g., human, in need thereof, an effective amount of a compound of formula (I) or a salt, ester, solvate, N-oxide, or prodrug thereof, e.g. a salt thereof as defined in claim 1 to 13.

26. The method of claim 25, wherein the organ is a kidney, lung or liver.

27. Use of a compound of formula (I) or a salt, ester, solvate, N-oxide, or prodrug thereof, e.g. a salt thereof as defined in claim 1 to 13 for use in the manufacture of a medicament for reducing one or more of hydroxyproline content or collagen Type I mRNA expression in an organ.

28. The use of claim 27, wherein the organ is a kidney, lung or liver.


SEQUENCE LISTING (12 sequences)

SEQ ID NO: 1 - DNA, 20 bp, Artificial Sequence
  Primer sequence
gccatcaggaacctcgagaa

SEQ ID NO: 2 - DNA, 21 bp, Artificial Sequence
  Primer sequence
ggagcatcatcaccagcaaag

SEQ ID NO: 3 - DNA, 21 bp, Artificial Sequence
  Primer sequence
ctcatactgataggagagacg

SEQ ID NO: 4 - DNA, 20 bp, Artificial Sequence
  Primer sequence
tcgaacttgagtttgaagtg

SEQ ID NO: 5 - DNA, 20 bp, Artificial Sequence
  Primer sequence
cgattccgtgggtggtggtg

SEQ ID NO: 6 - DNA, 24 bp, Artificial Sequence
  Primer sequence
catgccagagtctcgttcgttatc

SEQ ID NO: 7 - DNA, 19 bp, Artificial Sequence
  Primer sequence
agatcaagatcattgcccc

SEQ ID NO: 8 - DNA, 20 bp, Artificial Sequence
  Primer sequence
ttcatcgtattcctgtttgc

SEQ ID NO: 9 - DNA, 21 bp, Artificial Sequence
  Primer sequence
aagcaggctaatactgatagg

SEQ ID NO: 10 - DNA, 20 bp, Artificial Sequence
  Primer sequence
tgttgaaaagtagttctggg

SEQ ID NO: 11 - DNA, 20 bp, Artificial Sequence
  Primer sequence
cagaaggatgtaaaggatgg

SEQ ID NO: 12 - DNA, 20 bp, Artificial Sequence
  Primer sequence
tatttcttcttggacacacc